(19) United States
(12) Patent Application Publication
(10) Pub. No.: US 2019/0388057 A1
(43) Pub. Date: Dec. 26, 2019

(54) SYSTEM AND METHOD TO GUIDE THE POSITIONING OF A PHYSIOLOGICAL SENSOR

(71) Applicant: Cardio Square Inc., Richmond Hill (CA)

(72) Inventors: Qinghua Shen, Toronto (CA); Chi-Ming Chow, Toronto (CA); Steven Henry Fyke, Richmond Hill (CA); Oleg Michailovich, Waterloo (CA); Vene Evangelista, Toronto (CA)

(21) Appl. No.: 16/449,692

(22) Filed: Jun. 24, 2019

Related U.S. Application Data
(60) Provisional application No. 62/689,090, filed on Jun. 23, 2018.

(51) Int. Cl. A61B 8/00; G06T 7/73

(52) U.S. Cl. CPC A61B 8/4245; A61B 8/46; A61B 8/461; G06T 2207/20081; G06T 2207/10132; G06T 2207/20084; G06T 7/73

(57) ABSTRACT

Described is a method for providing positioning suggestions to the user of a physiological sensor and a kit for configuring an ultrasound system to provide positioning suggestions. The method includes steps of receiving an ultrasound image from an ultrasound probe, processing the ultrasound image to identify a set of features, determining from the set of features whether the ultrasound probe is correctly aligned, and determining an action suggestion to be given to a user of the ultrasound probe to improve its alignment. The kit includes a display monitor and a real-time video processor for interfacing with the ultrasound system to receive an ultrasound image from an ultrasound probe, process the ultrasound image to determine a set of positioning information, produce a set of positioning suggestion information for adjusting the positioning of the ultrasound probe, and transmit the set of positioning suggestion information to the display monitor for display.

### FIELD OF THE INVENTION

The present specification relates generally to physiological sensors, and specifically to a system and method to guide the positioning of a physiological sensor.

### BACKGROUND OF THE INVENTION

Echocardiography and other non-invasive imaging systems are often the first systems used in diagnostic imaging, such as imaging a heart or other internal organ. Echocardiography systems in particularly are used as they are often portable, non-invasive, and readily available.

Many medical and residency programs curriculums teach the use of point of care ultrasound (POCUS), such as for use in internal medicine, emergency care, and critical care. As a result, there are many novice echo users who need to be trained in acquiring proper echo views. The echocardiography images obtained by novice users may suffer from suboptimal image quality and inconsistency, affecting the diagnostic value of the images.

Many novice and experienced users of ultrasonic technology may appreciate feedback on their positioning of an ultrasonic probe.

### SUMMARY OF THE INVENTION

In an embodiment of the present invention, there is provided a method for providing positioning suggestions, comprising receiving an ultrasound image from an ultrasound probe; processing the ultrasound image to identify a set of features; determining from the set of features whether the ultrasound probe is correctly aligned; and determining an action suggestion to be given to a user of the ultrasound probe to improve the alignment of the ultrasound probe.

In an embodiment of the present invention, there is provided a kit for configuring an ultrasound system to provide positioning suggestions, comprising a real-time video processor for interfacing with the ultrasound system to receive an ultrasound image from an ultrasound probe of the ultrasound system, process the ultrasound image to determine a set of positioning information, produce a set of positioning suggestion information for adjusting the positioning of the ultrasound probe, and transmit the set of positioning suggestion information for display to a kit user; and a display monitor for receiving the set of positioning suggestion information from the real-time video processor and displaying the set of positioning suggestion information to the kit user.

### BRIEF DESCRIPTION OF THE DRAWINGS

The principles of the invention may better be understood with reference to the accompanying figures provided by way of illustration of an exemplary embodiment, or embodiments, incorporating principles and aspects of the present invention, and in which:

FIG. 1 is a workflow diagram of a method for providing positioning suggestions, according to an embodiment;

FIG. 2 is an example of an ultrasound guidance image, according to an embodiment;

FIG. 3 is an example of an ultrasound guidance image, according to an embodiment;

FIGS. 4A and 4B are example ultrasound images;

FIG. 5 is an example showing segmentation and rotation mapping information, according to an embodiment; and

FIG. 6 is a schematic diagram of a system for providing positioning suggestions, according to an embodiment.

Like reference numerals indicated like or corresponding elements in the drawings.

### DETAILED DESCRIPTION OF THE EMBODIMENTS

The description that follows, and the embodiments described therein, are provided by way of illustration of an example, or examples, of particular embodiments of the principles of the present invention. These examples are provided for the purposes of explanation, and not of limitation, of those principles and of the invention. In the description, like part are marked throughout the specification and the drawings with the same respective reference numerals. The drawings are not necessarily to scale and in some instances proportions may have been exaggerated in order more clearly to depict certain features of the invention.

Systems have been developed to classify ultrasound images and to assist users in capturing diagnostically relevant images. For example, it has been suggested that convolution neural networks (CNN) can be used to classify standard echocardiogram views and to determine echocardiogram image quality (see for example Madani, A., Arnaout, R., Mofrad, M. and Arnaout, R., 2018. Fast and accurate view classification of echocardiograms using deep learning. npj Digital Medicine, 1(1), p. 6 and Abdi, A. H., Luong, C., Tsang, T., Allan, G., Nouranian, S., Jue, J., Hawley, D., Fleming, S., Gin, K., Swift, J. and Rohling, R., 2017, February. Automatic quality assessment of apical four-chamber echocardiograms using deep convolutional neural networks. In Medical Imaging 2017: Image Processing (Vol. 10133, p. 101330S). International Society for Optics and Photonics, both hereby incorporated by reference), however such systems often check image quality after images have been taken and without pointing out what is wrong with the images. In another example, it has been suggested that a system using a camera and an ultrasonic probe outfitted with a barcode may be used to help users of the probe position the probe correctly for a diagnostically useful image (see for example Butterfly Network, “Augmentation Reality Acquisition software with Butterfly iQ”, accessed via https://www.youtube.com/watch?v=dlIOTFyKMVU, hereby incorporated by reference), however requiring that an operator hold a camera directed at the chest of a patent and at an operated probe may make the system inconvenient to use in practice. Often, sonographers and cardiology fellows are trained to use one hand to hold and position a probe while using their other hand to adjust image acquisition parameters such as acquisition mode, brightness, and depth of focus, to more quickly acquire an image having a desired image quality.

A variety of other improvements to image acquisition technologies have also been suggested. For example, methods have been suggested for reducing speckle (see for example Manzoor Razaak, Maria G. Martini, “Medical image and video quality assessment in e-health applications and services”, e-Health Networking Applications & Services (Healthcom) 2013 IEEE 15th International Conference on, pp. 6-10, 2013, hereby incorporated by reference). In another example, the automated measurement of ejection fraction has also been suggested (see for example Guppy-Coles, K., Prasad, S., Hillier, S., Smith, K., Lo, A., Sippel, J., Biswas, N., Dahiya, A. and Atherton, J., 2015. Accuracy of an operator-independent left ventricular ejection fraction quantification algorithm (Auto LVQ) with three-dimensional echocardiography: a comparison with cardiac magnetic resonance imaging. Heart, Lung and Circulation, 24, pp. S319-S320, hereby incorporated by reference). A survey of other ultrasound image quality-related suggestions is provided in Sumeet Gandhi, Wassim Mosleh, Joshua Shen, Chi-Ming Chow, “Automation, Machine Learning and Artificial Intelligence in Echocardiography: A Brave New World”, journal: Echocardiography, to be published July 2018, hereby incorporated by reference.

An aspect of this description relates to an artificial intelligence (AI) powered real-time assistant tool to help users acquire standard echo views with consistent image quality. A further aspect of this description relates to an artificial intelligence (AI) powered real-time assistant tool to help users acquire standard echo views in a shorter period of time than would otherwise be practical.

A yet further aspect of this description relates to recognizing a current ultrasound view. Another aspect of this description relates to determining the position of a current view relative to a desired standard view. A further aspect of this description relates to giving suggestions to a user on how to adjust an ultrasound probe position to a more desirable position. Another aspect of this description relates to giving suggestions to a user via feedback, such as visual feedback, audio feedback, and haptic feedback. A yet further aspect of this description relates to hardware and software to enable existing medical facility ultrasound machines, such as workstations and laptop-based machines, to provide a guidance feature. Another aspect of this description relates to hardware and software to enable hand held ultrasound machines, such as table and smart phone-based machines, to provide a guidance feature.

While the following description of an image acquisition guidance method and system focuses on echocardiogram applications of the method and system, the method and system in other embodiments could be used for other ultrasound applications and other image acquisition needs. Echocardiograph technology is used as an example here since the structure of a heart is one of the most complicated three-dimensional human organs.

Additionally, while the method and system could be used with a variety of echocardiogram technologies, methods, and views such as parasternal views, subcostal views, suprasternal views, stress echoes, contrast echoes, and transesophageal echoes, the following description focuses on the use of the method and system to image an apical chamber view in a transthoracic echocardiogram.

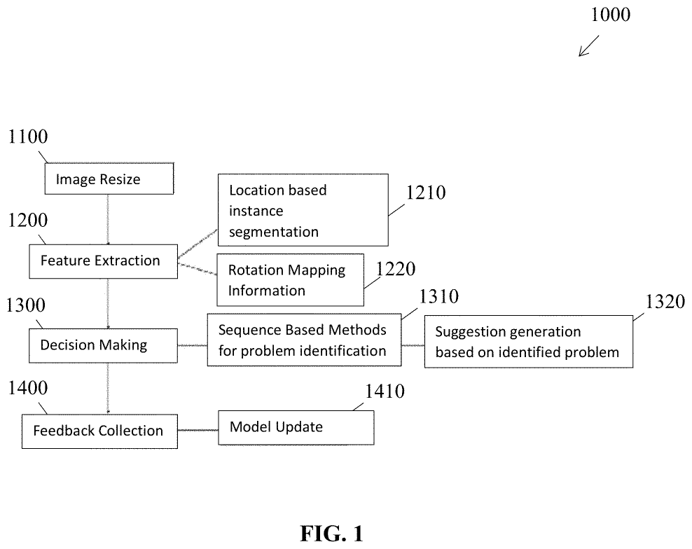

FIG. 1 depicts a method 1000 of guiding a user in acquiring an echocardiograph image. When a user has acquired a raw echo image, at step 1100 the raw echo image is reduced in size if method 1000 does not require a resolution as high as is provided by the raw echo image. For example, a raw echo image may be 1024 by 768-pixel image. Smaller images allow for faster processing time and better enable real-time processing. In some embodiments, a guidance method and system is able to provide reliable guidance using images having a resolution lower than 1024 by 768-pixel resolution, such as using 128 by 128-pixel images.

Step 1100 may be implemented using a variety of image resizing options, such as skip pixel, average grid, and Gaussian blur options. In some embodiments echo images are also set to black and white to further reduce computation costs, as many raw echo images are colored but only structural information is needed.

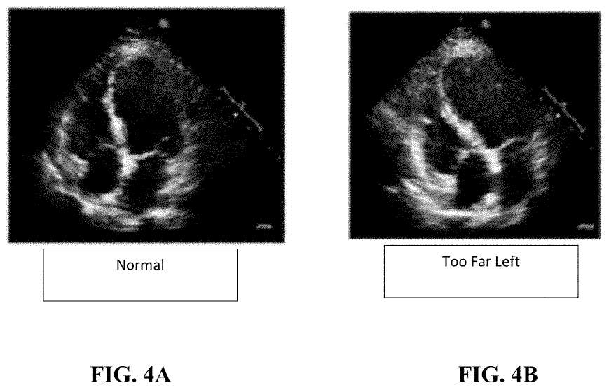

At step 1200 features of the echo image are extracted. A variety of view or feature identification options are available. For example, convolutional neural networks (CNN) having multiple stacked convolutional, activation, and pooling layers have been used extract features from images, however such networks can have difficulty identifying transition, rotation, and shifting. For example, FIG. 4A shows a normal heart echo image while FIG. 4B shows a heart echo image captured while the heart was rotated with respect to the probe. While the images of FIGS. 4A and 4B are almost the same, the image of FIG. 4B is considered incorrect and a user acquiring the image would need to be told to adjust the probe.

The echo image feature and view identification of step 1200 needs to be sensitive to variations such as rotation as well as being able to identify features in the image. Additionally, the echo image feature and view identification of step 1200 needs to be sensitive to natural variations in the arrangement of components of organs such as the heart. For example, the structure of the left ventricular and right ventricular of a patient with dextrocardia is mirrored compared to normal heart structure. For proper image problem identification, step 1200 needs to be able to tolerate acceptable variations in images.

In the embodiment method of FIG. 1, step 1200 may incorporate one or more known view and feature identification options, but also includes a location-based segmentation sub-step 1210 and a rotation mapping sub-step 1220.

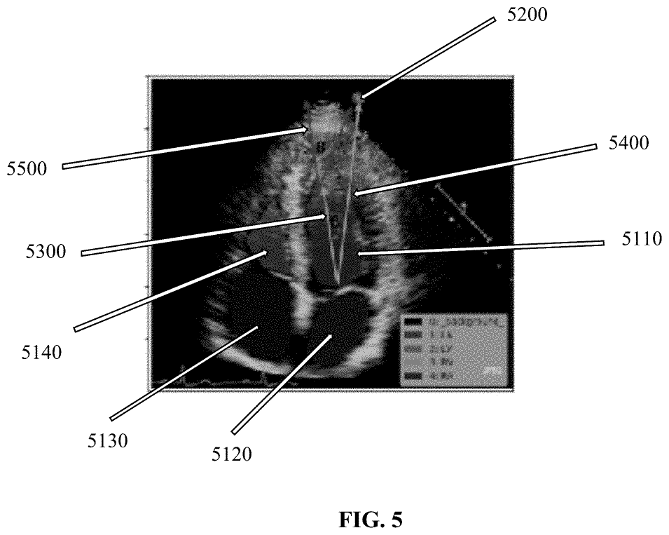

Segmentation sub-step 1210 may employ U-net and mask r-cnn options (see for example Ronneberger, O., Fischer, P. and Brox, T., 2015, October. U-net: Convolutional networks for biomedical image segmentation. In International Conference on Medical image computing and computer-assisted intervention (pp. 234-241). Springer, Cham and He, K., Gkioxari, G., Dollar, P. and Girshick, R., 2017, October. Mask r-cnn. In Computer Vision (ICCV), 2017 IEEE International Conference on (pp. 2980-2988). IEEE, both herein incorporated by reference). As indicated in the image shown in FIG. 5, segmentation sub-step 1210 identifies different components of the body shown in the echo image, such as using a multi-classes version of U-net. In some embodiments, components identified in a segmentation sub-step are visually identified to a user, such as first segment 5110, second segment 5120, third segment 5130, and fourth segment 5140 shown in FIG. 5. In some embodiments, the location of each component is also captured, such as by approximating the boundary of each component as a rectangular box and using the central point of the box to denote the location of the component.

Rotation mapping sub-step 1220 uses the mark point in an echo image as an anchor to measure rotation information and uses information about features and components shown in an echo image as identified in the segmentation sub-step 1210. The mark point, such as mark point 5200 of FIG. 5, is included in echo images to indicate the location of the probe, making it an excellent pick as a reference point for measuring rotation information.

FIG. 5 indicates how a rotation mapping sub-step 1220 could be performed. FIG. 5 shows an example of measuring the rotation of a left ventricular (LV), the LV being identified in FIG. 5 as first segment 5110. The long axis 5300 of the LV is first determined, then the distances and angles between the long axis and two vectors is calculated. The first of the two vectors 5400 starts at the remote point of the long axis of the LV, and the second of the two vectors 5500 starts at the near side point of the long axis of LV. Both vectors end at the mark point 5200. The determination of which end of the long axis is the remote end and which is the near side end is determined relative to the mark point. In other embodiments, other angles could also be used to determine the rotation with reference to the mark point. Provided the calculation method is consistent for all heart components, the rotation map is useful to determine relative rotation among different components.

It has been suggested that rotation mapping information could also be found using neural networks, such as the capsule network (see for example Sabour, S., Frosst, N. and Hinton, G. E., 2017. Dynamic routing between capsules. In Advances in Neural Information Processing Systems (pp. 3859-3869), hereby incorporated by reference). However, training a capsule network often requires a large amount of date and it may be difficult to use a capsule network to provide rotation mapping information relative to a mark point.

At step 1300, a decision regarding whether the probe is correctly positioned. Step 1300 includes identifying problems at step 1310 and generating suggestions at step 1320. Using a knowledge of required heart components, such as identified by the American Society of Echocardiography (ASE) guidelines on echo image quality, and a knowledge of proper rotation and orientation, at step 1310 the uses the results of step 1200 to determine if any components of the heart that should be shown are not shown. At step 1320 suggestions are made, such as to slide, angle, or sweep a probe to find the required components or orientation. Step 1320 can also calculate the direction or rotation for the slide, angle, or sweep motions and include those details in the suggestion as well. Step 1320 compares what is shown in an echo image to the structure of the heart, such as the structure defined or identified by the ASE, and makes suggestions to guide the user of a probe in how to shift the position of the probe.

For example, if the right ventricular (RV) and right atrium (RA) shown in FIG. 5 had been missing, at step 1300 knowledge of ideal locations and rotation orientation could be used to find that the current distances of the two vectors identified in step 1200 are larger than usual and define larger angles. At step 1320 it may be suggested that the user adjust the angle or position of the probe to reduce both the distances and the angles. Other scenarios may follow a similar analysis as that set out in the above example.

To provide accurate suggestions, a sequence of images could be used in some embodiments, particularly where echo images are of poor quality. For example, a majority voting scheme could be employed, where for a set of 2N+1 consecutive images the method may be employed to determine a set of suggested actions with one suggestion for each image, and the suggestion most often resulting would be recommended to the user. In the event of a tie of two or more suggestions, the voting scheme will require a further round of voting for the top candidates only to select the majority winner.

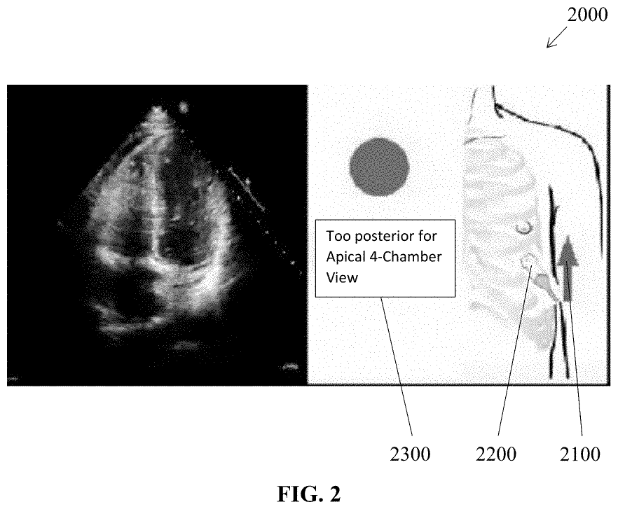

A specific example of a suggestion is shown in FIG. 2. FIG. 2 shows a composite image or interface 2000 which includes an echo image on the left and a schematic diagram on the right. The right-side schematic diagram displays visual feedback to help a user understand the probe adjustment suggestions. Arrow 2100 is provided to indicate an adjustment suggestion to direct the user of probe 2200 regarding the adjustment of probe 2200. Interface 2000 also includes a textual indication 2300 of the problem identified, to give the user an indication of why the suggestion indicated by arrow 2100 is being suggested.

At step 1400, method 1000 collects feedback. The decisions and suggestions resulting from the neural network or other parts of the method are recorded, along with the actual movements made and how actual movements affected the echo images available. This information is used to update the network, for example through machine learning, neural network backpropagation, or other AI updating. For example, if the actual movement made is not as suggested but does result in a standard view being acquired, this information can be recorded and used to update future suggestions at step 1410. In some embodiments a feedback collection step is simply a collection of information or may be excluded from a method or system altogether.

Of particular value to some embodiments is the explanation provided with an action suggestion to explain why the action suggestion was suggested. For example, such suggestions may help novice users learn how to acquire standard echo views faster and may help users apply personal experience to determine whether a suggestion makes sense. Some embodiments may include an expert mode function, which may be used to make suggestions to an experienced user as an assist to the user's own experience, rather than a full replacement; such a mode in particular may assist a system in training itself by learning from the actions of experts in accepting or ignoring suggestions.

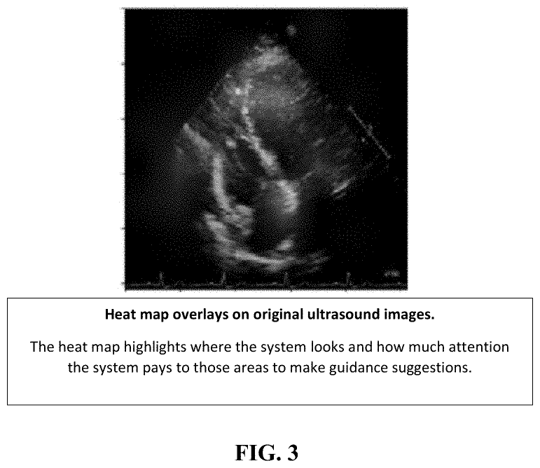

FIG. 3 indicates an example image display mode, providing an explanation of why a suggestion was made to a user. Rather than show all the angles and distances determined using a method such as method 1000, the image shows highlighting via a heat map to show which part of the image were most important in informing a suggestion. The echo image of FIG. 3 is a view which is too far right for an apical four chamber view, which was determined mainly with reference to the shape and rotation of the apex and septum of the imaged heart. Two other parts of the echo image particularly considered were the right-hand side boundary of the heart wall and the left-hand side void space. The heat map applied in FIG. 3 was a result of considering the weights of the neural network used to analysis the echo image (see for example Selvaraju, R. R., Das, A., Vedantam, R., Cogswell, M., Parikh, D. and Batra, D., 2016. Grad-CAM: Why did you say that?. arXiv preprint arXiv:1611.07450, hereby incorporated by reference).

Methods such as method 1000 can be applied to a variety of echo image acquisition systems which incorporate a screen and sufficient computing power. Implementation system which include a graphic processing unit (GPU) may be particularly able to process the images quickly enough to make real-time guidance available. Advances in computing ability, such as improvements in GPU design, will increasingly make real-time processing feasible.

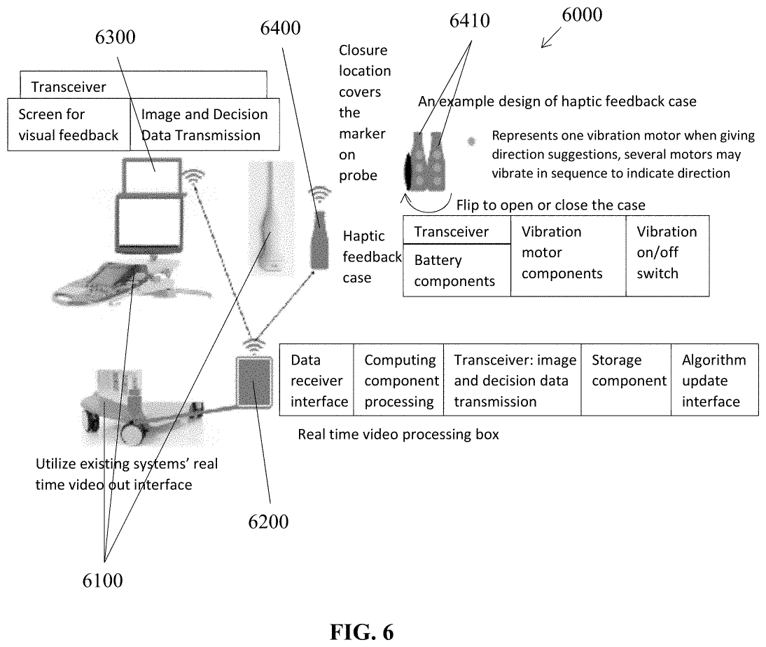

FIG. 6 shows a system 6000 for providing guidance suggestions. System 6000 includes a standard ultrasound imaging system 6100, such as are commonly used in medical faculties. System 6000 also includes a real-time video processing box 6200, a monitor 6300, and a haptic feedback case 6400. In many cases, commonly used ultrasound imaging systems, such as system 6100, do not have sufficient computing power to practically run real-time image processing.

The system 6000 of FIG. 6 makes available to system 6100 the computing power and output device needed to permit system 6000 to provide guidance suggestions, using real-time video processing box 6200 and monitor 6300. In some embodiments the real-time video processing box 6200 and monitor 6300 are provided as a single plug-in box to be added to system 6100. The optional haptic feedback case 6400 may also be provided in some embodiments, to provide haptic feedback in addition to visual feedback.

The real-time video processing box 6200 includes a data receiver interface to connect to an existing systems' real-time video out interface to obtain echo images, a computing component to process images, and a transceiver component to send image and suggestion information to the monitor 6300 and to send movement information to the haptic feedback case 6400. Transmission of information may be wirelessly or via a cable. Real-time video processing box 6200 may also include a storage component, such as to store data for model updates, and an interface to allow a user to update software, such as an Internet connection component that can be used to exchange data with a cloud computing facility.

Monitor 6300 includes a transceiver component to receive data from the processing box 6200 and a screen for visual feedback. According to an embodiment, the monitor can be a computer screen, a smart glass with screen, or a virtual reality type of screen mounted on a user's head.

Haptic feedback case 6400 includes a transceiver to receive data from the processing box 6200, a battery or other power supply component, a set of vibration motor components, and a switch to allow the feedback mechanism to be turned on or off. The case 6400 may also include structure to ensure that the case is aligned with the mark line or point of the probe that it is provided to encase. In some embodiments, case 6400 is provided as a set of two half-cases 6410 which can be closed over a probe. Case 6400 is provided to help guide a user by providing vibration or other haptic feedback reflecting movement suggestions. The haptic feedback case 6400 is provided as an optional supplement to visual indication of suggestions.

Various embodiments of the invention have been described in detail. Since changes in and or additions to the above-described best mode may be made without departing from the nature, spirit or scope of the invention, the invention is not to be limited to those details but only by the appended claims.

What is claimed is:

1. A method for providing positioning suggestions, comprising:
receiving an ultrasound image from an ultrasound probe;
processing the ultrasound image to identify a set of features;
determining from the set of features whether the ultrasound probe is correctly aligned; and
determining an action suggestion to be given to a user of the ultrasound probe to improve the alignment of the ultrasound probe.

2. The method of claim 1, wherein processing the ultrasound image to identify a set of features includes:
segmenting the ultrasound image to identify components; and
mapping rotation information with reference to a mark point.

3. The method of claim 2, wherein processing the ultrasound image includes defining a segment, defining a long axis of the segment, and determining the distance and orientation of the long axis relative to the mark point.

4. The method of claim 3, wherein processing the ultrasound image includes processing the ultrasound image using a neural network trained on a set of relevant human organ data.

5. The method of claim 1, further comprising, prior to processing the ultrasound image, reducing an image file size of the ultrasound image.

6. The method of claim 1, wherein the action suggestion is one of a tilt, sweep, rotate, slide, rock, or angle motion.

7. The method of claim 1, further comprising storing the action suggestion to allow the method to accumulate a set of action suggestions to be used together to determine how to direct the user of the ultrasound probe.

8. The method of claim 1, further comprising presenting the action suggestion to the user.

9. The method of claim 8, wherein presenting the action suggestion to the user comprises presenting a visual action direction.

10. The method of claim 8, wherein presenting the action suggestion to the user comprises presenting a haptic action direction.

11. The method of claim 8, further comprising, after presenting the action suggestion:
detecting a movement of the ultrasound probe; and
collecting a further ultrasound image from the ultrasound probe to be used in reviewing the effectiveness of the processing step and the determining step.

12. The method of claim 11, wherein collecting the further ultrasound image includes collecting a compliance indication, the compliance indication indicating whether the action suggestion was followed.

13. A kit for configuring an ultrasound system to provide positioning suggestions, comprising:
a real-time video processor for interfacing with the ultrasound system to receive an ultrasound image from an ultrasound probe of the ultrasound system, process the ultrasound image to determine a set of positioning information, produce a set of positioning suggestion information for adjusting the positioning of the ultrasound probe, and transmit the set of positioning suggestion information for display to a kit user; and
a display monitor for receiving the set of positioning suggestion information from the real-time video processor and displaying the set of positioning suggestion information to the kit user.

14. The kit of claim 13, further comprising a haptic feedback case for fitting over the ultrasonic probe, receiving a set of haptic feedback information from the real-time video processor, and providing haptic feedback to the kit user.

15. The kit of claim 13, wherein the real-time video processor includes an algorithm update interface.
